(19) United States
(12) Patent Application Publication
(10) Pub. No.: US 2007/0264710 A1
(43) Pub. Date: Nov. 15, 2007

(54) Process for Purifying Human Thrombopoietin with High Content of Sialic Acid

(76) Inventors: Hyea-Kyung Ahn, Kyunggi-do (KR); Joo-Young Chung, Kyunggi-do (KR); Young-Ju Cho, Kyunggi-do (KR); Tae-Soo Kim, Kyunggi-do (KR); Yeo-Wook Koh, Kyunggi-do (KR); Seung-Wook Lim, Jyunggi-do (KR); Yeon-Joo Hong, Kyungsangbuk-do (KR)

Correspondence Address: MEDLEN & CARROLL, LLP, 101 HOWARD STREET, SUITE 350, SAN FRANCISCO, CA, 94105, US

(21) Appl. No.: 10/573,359

(22) PCT Filed: Oct. 9, 2003
(86) PCT No.: PCT/KR03/02079

§ 371 Date: Mar. 5, 2007

(51) Int. Cl. C07K 1/00; C12N 5/06

(52) U.S. Cl. 435/360; 530/413

(57) ABSTRACT

Disclosed is a process for producing a human thrombopoietin (hTPO)-containing culture fluid, comprising the step of culturing a eukaryotic cell expressing hTPO in a serum-free medium that contains a negligible amount of serum. In addition, the present invention discloses a process for purifying hTPO from a hTPO-containing biological fluid, comprising the steps of (a) subjecting the biological fluid to affinity chromatography; (b) subjecting an eluate obtained at step (a) to hydrophobic interaction chromatography; (c) subjecting an eluate obtained at step (b) to reverse phased chromatography; and (d) subjecting an eluate obtained at step (c) to anion exchange chromatography. In addition, the present invention discloses hTPO with a high sialic acid content obtained by the process which comprises the step of loading, the eluate obtained at step (c) onto an ionic exchange chromatography column and collecting hTPO with a high content of sialic acid eluted selectively from the column by a 0.15-0.3M sodium chloride gradient.

### TECHNICAL FIELD

The present invention, in general, relates to a process for producing a culture fluid containing human thrombopoietin (hTPO) and a process for purifying hTPO from the culture fluid. More particularly, the present invention relates to a process for isolating and purifying hTPO with a high content of sialic acid from a biological fluid containing hTPO.

### PRIOR ART

A platelet growth factor, that is, thrombopoietin (TPO) is known as a cytokine regulating blood platelet counts (Lok et al., Nature, 369: 565-568 (1994); and De savage, F. J. et al., Nature, 369: 533-568 (1994)). TPO, which is a glycoprotein synthesized and secreted in the liver and kidney, functions to stimulate proliferation and differentiation of megakarocyte precursors, and induces the maturation of megakaryocytes to platelets.

Currently, the most common means of treating thrombocytopenia is by platelet transfusion. However, such a therapy is difficult because of shortages of platelet transfusion donors, bleeding as a side effect, platelet's contaminated with various viruses and platelet's antigenicity. The platelet growth factor, TPO, is believed to be applicable in treating a variety of diseases associated with platelets, while reducing the adverse effects caused by platelet transfusion. hTPO cDNA was first cloned in 1994, and widely published in papers and patents (Lok et al., Nature, 369: 565-568 (1994); De savage, F. J. et al., Nature, 369: 533-568 (1994); Miyazaki et al., Experimental hematol., 22: 828 (1994); and International Pat. Publication WO95/18858). hTPO specifically acts on the platelet precursors, progenitor (colony-forming) cells in bone marrow, and stimulates proliferation and differentiation of megakaryocytes, the platelet precursors, resulting in increased platelet production. Due to such functions, hTPO is effective in the treatment of thrombocytopenia caused by situations such as anticancer therapy and bone marrow transplantation. In clinical trials, hTPO remarkably increased platelet counts and showed mild side effects, and thus is a candidate for a novel drug (Shinjo et al., Leukemia, 12: 195-300 (1998); and Martin et al., J. Pediatr. Hematol. Oncol., 20(1): 36-43 (1998)). Actually, Genentech Inc. prepared a hTPO (International Pat. Publication WO95/18868), and continued Phase III clinical trials in collaboration with Pharmacia & Upjohn. Kirin are also conducting clinical trials of hTPO analogues (International Pat. Publication WO95/21919). The present inventors invented hTPO analogues with higher in vivo biological activity than wild type hTPO (International Pat. Publication WO99/00347), which are expected as excellent therapeutic agents for thrombocytopenia.

Large scale production of hTPO was accomplished by using the cells transfected with the expression vector for hTPO with genetic recombination technology. In this way, hTPO is purified from the culture fluid after culturing the transformed cells in serum-containing medium, and used in the medical field. However, when the transformed cells are cultured in serum-containing medium, animal-derived factors may give rise to adverse effects.

Therefore, there is an urgent need for development of methods of producing/purifying hTPO with a high purity suitable for medical uses, without risk of contamination with microorganisms or impurities and with a high activity.

The present inventors found that, when eukaryotic cells transformed with an hTPO-expressing expression vector are cultured in a serum-free medium that contains a negligible amount of serum, adverse effects by animal-derived factors (e.g., viruses) are minimized, and hTPO is obtained at a high expression efficiency.

It is therefore an object of the present invention to provide a process for producing hTPO by culturing a eukaryotic cell expressing hTPO in a serum-free medium.

In addition, the present inventors successfully purified hTPO with a high purity suitable for pharmaceutical uses by applying a biological fluid containing hTPO to a series of chromatographies (affinity chromatography, hydrophobic interaction chromatography, reverse phased chromatography and anion exchange chromatography).

It is therefore another object of the present invention to provide a process for purifying hTPO with a high purity from a biological fluid containing hTPO.

Most sugar chains in many glycoproteins used as therapeutic agents have a critical role in the biological activity of the glycoproteins (Takeuchi et al., Proc. Natl. Acad. Sci. USA. 86: 7819 (1989)). In case of erythropoietin (EPO), the numbers and sugar types of glycosylation affect stability and solubility of EPO, especially, the content of sialic acid are important for extending in vivo half life of EPO. In this regard, when selecting a host cell for preparation of a recombinant glycoprotein, its glycosylation ability should be preferentially considered. It was reported that glycoprotein with a high content of sialic acid can be purified by anion exchange chromatography based on the negative charge of sialic acid (Glycoconj J., 13(6): 1013-20 (1996)). However, in case of TPO, there is still no report of a relationship between sialic acid content and its in vivo biological activity. The present inventors found that, when hTPO with different sialic acid contents were purified by a chromatography method according to the present invention, the hTPO activity was increased in proportion to the content of sialic acid.

It is therefore a further object of the present invention to provide hTPO with a high content of sialic acid, thus resulting in improved in vivo biological activity by using various chromatography steps.

### DISCLOSURE OF THE INVENTION

The present invention relates to a process for producing a hTPO-containing culture fluid, comprising the steps of culturing eukaryotic cells expressing hTPO in a 3-6.5% serum-containing medium, subsequently culturing the cell in a 0.5-1.5% serum-containing medium and then culturing the cell in a serum-free medium that is substantially free from serum.

The eukaryotic cell is preferably a Chinese hamster ovary cell line (CHO), and more preferably, selected from the group consisting of CHO dhfr-/pD40434 (KCTC 0630BP), CHO dhfr-/pD40449 (KCTC 0631BP) and CHO dhfr-/pD40458 (KCTC 0632BP). The eukaryotic cell is also inoculated in a 0.5-1.5% serum-containing medium at a density of 1.0×10⁴ to 1.0×10⁶ cells/ml, and preferably, at a density of 1.5×10⁵ cells/ml. The serum-free medium is preferably complemented with butyric acid and yeastolate.

In addition, the present invention relates to a process for purifying hTPO from a hTPO-containing biological fluid, comprising the steps of (a) subjecting the biological fluid to affinity chromatography; (b) subjecting the eluate obtained at step (a) to hydrophobic interaction chromatography; (c) subjecting the eluate obtained at step (b) to reverse phased chromatography; and (d) subjecting the eluate obtained at step (c) to anion exchange chromatography.

Preferably, at step (d), the eluate obtained at step (c) is loaded onto an ion exchange chromatography column, and hTPO eluted selectively from the column by a 0.15-0.3M sodium chloride gradient is collected. In addition, the process preferably may comprise a step of carrying out gel filtration chromatography after step (d). The hTPO-containing biological fluid is preferably a culture fluid obtained by culturing a eukaryotic cell transformed with an hTPO-expressing vector in a serum-free medium. At step (a), a column used in the affinity chromatography is preferably eluted with a phosphate buffer containing 1 M of sodium chloride. At step (c), a column used in the reverse phased chromatography is preferably eluted by ethanol gradient.

Further, the present invention relates to a hTPO-containing fraction obtained by the process.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flowchart showing a process for producing hTPO by a serum-free culture using a cell factory and subsequent chromatography;

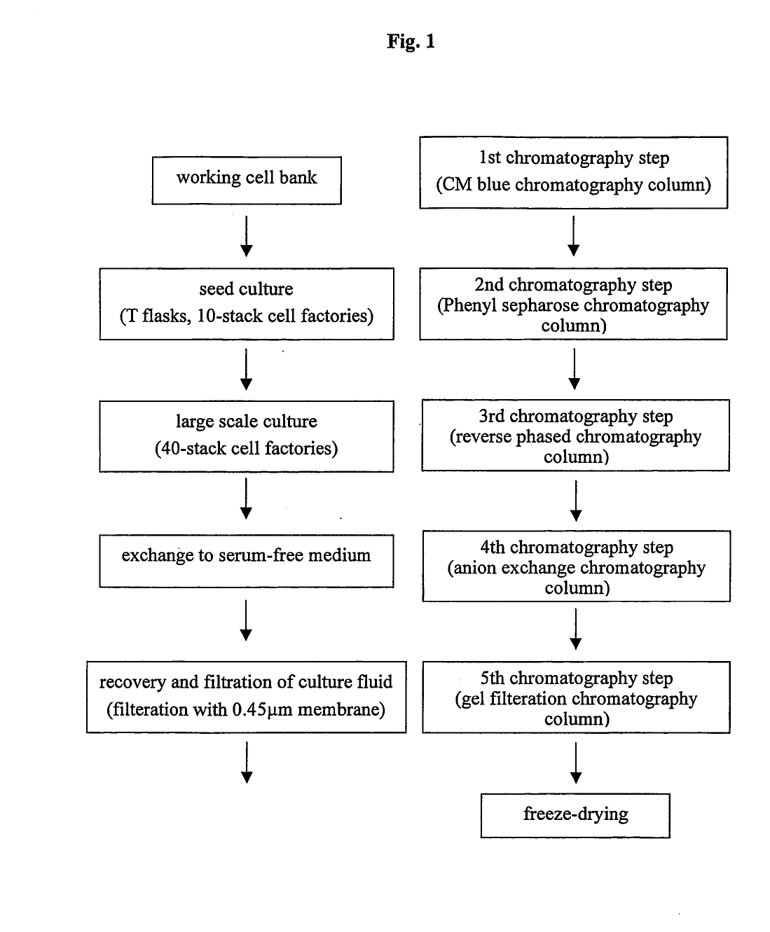

FIG. 2 shows expression levels of a hTPO analogue during a serum-free culture using cell factories;

FIG. 3a shows a result of the Coomassie blue staining of a SDS-polyacrylamide gel on which purified hTPO has been separated, and FIG. 3b shows Western blotting analysis of the gel;

FIG. 4 shows a result of a reverse phased HPLC, demonstrating that a purified hTPO analogue has a purity of over 99%;

FIG. 5 shows a result of size exclusion HPLC, demonstrating over 98% of a purified hTPO analogue exists in monomer form;

FIGS. 6a and 6b show pI values and sialic acid contents of hTPO-containing fractions by isoelectrofocusing analysis;

FIGS. 7a and 7b show the in vivo biological activity of a hTPO analogue according to its sialic acid content;

FIG. 8 shows the in vivo biological activity of a hTPO analogue in high sialic acid-fractions; and

FIG. 9 shows the expression levels of a hTPO analogue according to various ingredients contained in a serum-free medium.

### BEST MODES FOR CARRYING OUT THE INVENTION

Most cell surface proteins and secretory proteins produced in eukaryotic cells are modified by one or more oligosaccharide groups. Such modification is called glycosylation, and oligosaccharides are attached to specific sites on the backbone of a polypeptide. Two glycosylation patterns are known. One is O-linked glycosylation, in which an oligosaccharide is linked to a serine or threonine residue, and the other is N-linked glycosylation, in which an oligosaccharide is linked to asparagine (Asn) residue. N-linked glycosylation occurs at a specific amino acid sequence, particularly, Asn-X-Ser/Thr, wherein X is any amino acid excluding proline. N-linked oligosaccharide has a structure distinct from O-linked oligosaccharide, and sugar chains found in the N-linked type also differ from the O-linked type. A sugar residue found in both O-linked oligosaccharides and N-linked oligosaccharides is a member of the sialic acid family. “Sialic acid” is a common name for about 30 native acidic carbohydrates that are essentially found in numerous sugar moieties (Society Transactions, 11, 270-271 (1983)). The most frequently found sialic acid is N-acetylneuramic acid, and the second is N-glycolylneuramic acid (Schauer, Glycobiology, 1, 449-452 (1991)).

Wild type hTPO is a glycoprotein, which is expressed as a precursor consisting of 353 amino acids in the cell and secreted in an active form of 332 amino acids to the extracellular space after a signal peptide of 21 amino acids is cleaved from the precursor. hTPO analogues may have a different glycosylation pattern from the wild type hTPO. Representative examples of the hTPO analogues include a hTPO analogue prepared by introducing one or more N-linked glycosylations into hTPO of 174 amino acids with a deletion at the C-terminus through substitution of particular bases in a cDNA sequence encoding hTPO with a glycosylation motif sequence, Asn-X-Ser/Thr (X is any amino acid excluding proline) (International Pat. Publication WO96/25498); an hTPO analogue with an additional sugar chain, which is prepared by introducing a sugar chain into a full wild type hTPO form (H L Park et al., J. Biol. Chem., 273:256-261, 1998); and an hTPO analogue with N-linked glycosylation by substituting amino acid residues at position 164, 193, 157 and 164, 117 and 164, or 108 of wild type hTPO with asparagine (Korean Pat. Publication No. 2001-0078744).

The present inventors obtained an hTPO-containing culture fluid not contaminated with any serum-derived factor, by culturing a eukaryotic cell transformed with an hTPO-expressing vector in a 3-6.5% serum-containing medium, subsequently in a 0.5-1.5% serum-containing medium, and then in a serum-free medium that contains a negligible amount of serum. In this case, hTPO was produced with higher expression efficiency than in case of culture only in a serum-containing medium. In the conventional method using a low serum-containing medium, 5% or higher serum was contained in the medium. However, in the present invention, the culture started in the 3-6.5% serum-containing medium, subsequently performed in the 0.5-1.5% serum-containing medium, and finally completed in the serum-free medium that is substantially free from serum, thereby allowing production of hTPO while minimizing its contamination with serum-derived factors.

Therefore, in an aspect, the present invention provides a process for producing an hTPO-containing culture fluid, comprising the steps of culturing a eukaryotic cell expressing hTPO in a medium containing 3-6.5% serum, preferably, 4-6% serum, and more preferably, 5% serum; subsequently culturing the cell in a medium containing 0.5-1.5% serum, and preferably, 1% serum; and then culturing the cell in a serum-free medium that is substantially free from serum.

In the process for producing an hTPO-containing culture fluid, the eukaryotic cell expressing hTPO refers to a mammalian cell line capable of growing and surviving in monolayer culture or suspension culture using a culture medium containing suitable nutrients and growth factors. The growth factors essential for growth of a particular cell line, for example, as described in Mammalian Cell Culture, Mather, J. P. ed., Plenum Press, N.Y. (1984) and by Barnes and Sato, (1980) Cell, 22:649, may be determined easily by experimental experience without a heavy financial burden. The mammalian host cell suitable for the process of the present invention includes hTPO analogue-expressing transfected Chinese hamster ovary cells (CHO), COS cells, hybridoma cells, for example, mouse hybridoma cells, baby hamster kidney cells, 293 cells and mouse L cells. In particular, hTPO analogue-expressing CHO dhfr-/pD40434 (KCTC 0630BP), CHO dhfr-/pD40449 (KCTC 0631BP) and CHO dhfr-/pD40458 (KCTC 0632BP) are preferred. Of them, CHO dhfr-/pD40458 (KCTC 0632BP) is most preferred. In the culture using a 0.5-1.5% serum-containing medium, the hTPO-expressing cell is inoculated at a density of over 1×10⁴ cells/ml, preferably, 1×10⁴ to 1×10⁶ cells/ml, and more preferably, 1.5×10⁵ cells/ml.

In one embodiment, a hTPO analogue-containing culture fluid was obtained by culturing a CHO dhfr-/pD40458 cell line transformed with an hTPO analogue-expressing vector in a 3-6.5% serum-containing medium, subsequently, culturing the cells in a 0.5-1.5% serum-containing medium after inoculation at a density of 1×10⁴ to 1×10⁶ cells/ml, and then culturing the cells in a serum-free medium. Preferably, as described above, a culture supernatant from the hTPO analogue-containing culture fluid by a serum-free culture may be used as an hTPO analogue-containing biological fluid in the process for purifying hTPO according to the present invention.

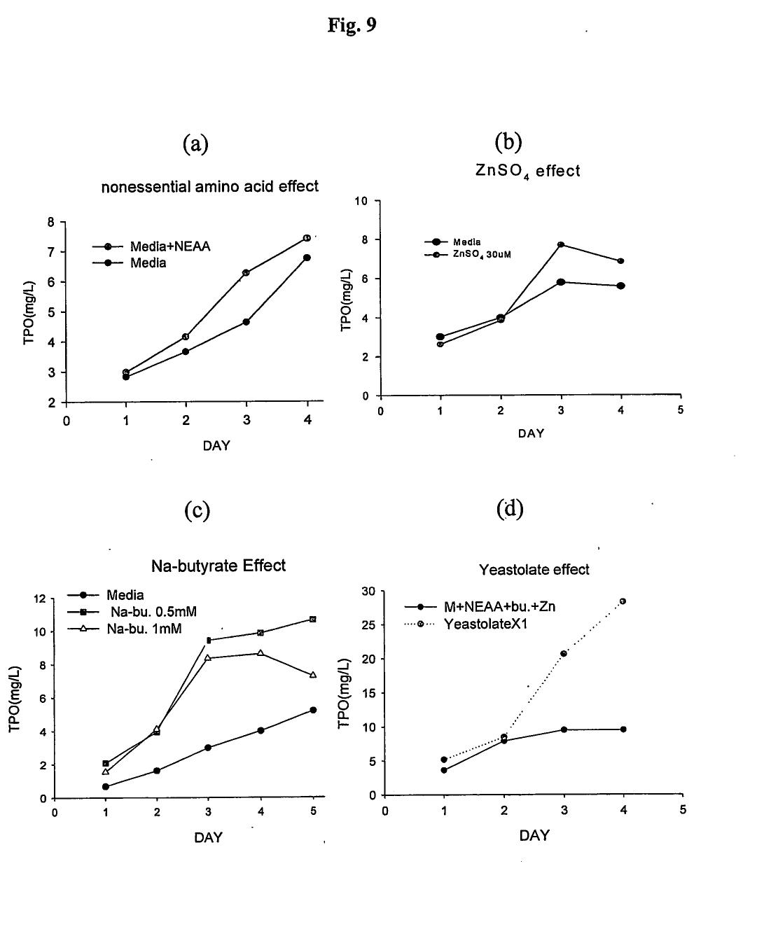

The term “serum-free medium”, as used herein, is intended to designate a nutrition medium which is substantially free from mammalian-derived serum (e.g., fetal bovine serum (FBS)). The term “substantially free from serum” means that a cell culture medium contains about less than 0.5% serum, and preferably, about 0-0.1% serum. As described above, the adverse effects caused by serum-derived factors, e.g., viruses, can be minimized by purifying hTPO from a culture fluid obtained by culturing an hTPO analogue-expressing eukaryotic cell in a serum-free medium that contains a negligible amount of serum. A nutrition medium for cell growth typically contains energy sources in forms of carbohydrate (e.g., glucose), all essential amino acids, vitamins, and/or other organic compounds, free fatty acids and trace elements that are required for cell growth in low concentrations (typically, organic compounds or natural elements required for cell growth in very low concentrations within a micromole), and may be supplemented with one or more selected from the group consisting of hormones and other growth factors (e.g., insulin, transferrin and epidermal growth factor), salts and buffers (e.g., calcium, magnesium and phosphate), nucleosides and bases (e.g., adenine, thymidine and hypoxanthine), and proteins and tissue hydrolysates. The present inventors investigated the effects of nonessential amino acids, ZnSO₄, sodium butyrate and yeastolate as an additive of serum-free medium on hTPO expression. As shown in FIG. 9, sodium butyrate added to the medium at 0.5 mM concentration was more effective than the case of being used at 1 mM concentration. The case of adding yeastolate to the medium showed a much higher hTPO expression level than the case of adding nonessential amino acids (NEAA), sodium butyrate and ZnSO₄ to the medium. In this regard, the present inventors used a serum-free medium supplemented with butyric acid and yeastolate. Compared with the use of a serum-containing medium, the use of a serum-free medium resulted in an increased hTPO expression while minimizing serum-derived impurities, thereby facilitating purification of the expressed hTPO.

In addition, the present inventors successfully purified hTPO with a high purity by chromatographically purifying hTPO from a hTPO-containing biological fluid. In detail, the present inventors found that hTPO with a high purity can be obtained by a process comprising the steps of (a) subjecting the biological fluid to affinity chromatography; (b) subjecting the eluate obtained at step (a) to hydrophobic interaction chromatography; (c) subjecting the eluate obtained at step (b) to reverse phased chromatography; and (d) subjecting the eluate obtained at step (c) to anion exchange chromatography. In particular, hTPO with a high content of sialic acid was obtained in a high purity form by a process comprising the steps of (a) subjecting an hTPO-containing biological fluid to affinity chromatography; (b) subjecting the eluate obtained at step (a) to hydrophobic interaction chromatography; (c) subjecting the eluate obtained at step (b) to reverse phased chromatography; and (d) subjecting the eluate obtained at step (c) onto an anion exchange chromatography column and collecting hTPO eluted selectively by a 0.15-0.3M sodium chloride gradient.

Therefore, in another aspect, the present invention provides a process for purifying hTPO from a hTPO-containing biological fluid, comprising the steps of (a) subjecting the biological fluid to affinity chromatography; (b) subjecting the eluate obtained at step (a) to hydrophobic interaction chromatography; (c) subjecting the eluate obtained at step (b) to reverse phased chromatography; and (d) subjecting the eluate obtained at step (c) to anion exchange chromatography. Preferably, at step (d), the eluate obtained at step (c) is loaded onto anion exchange chromatography column, and hTPO with a high content of sialic acid eluted selectively from the column by a 0.15-0.3M sodium chloride gradient. Preferably, the process may further comprise a step of subjecting an eluate obtained by anion exchange chromatography to gel filtration chromatography to remove aggregates.

In both the process for producing a hTPO-containing culture fluid and the process for purifying hTPO from the culture fluid, TPO, which is derived from human, contains wild type hTPO and its analogues. The hTPO analogues have biological activity more than wild type hTPO. The hTPO analogues comprise hTPO mutants with substitutions, insertions and deletions at some amino acid positions of the wild type hTPO, and may have a different glycosylation pattern from the wild type hTPO. As described above, the hTPO analogues may have increased glycosylations or sugar chains at new positions.

The term “biological fluid”, as used herein, may contain cells, constituents or metabolic products of the cells, or refer to all fluids derived from the cells. The biological fluid includes, but is not limited to, cell culture fluids, cell culture supernatants, cell lysates, cell extracts, tissue extracts, blood, plasma, serum, milk, urine and fractions thereof. If containing hTPO, one of the various biological fluids as described above may be used as a starting material in the process for purifying hTPO. Preferably, a culture supernatant obtained by the aforementioned serum-free culture is used.

In the process for purifying hTPO, the affinity chromatography is based on the specific interactions between biological molecules by reversible non-covalent bonding. That is, this chromatography method does not use a difference in physicochemical properties, but specificity of a binding system, in which a specific binding partner, what is called, ligand is covalently bound to typically an insoluble matrix (e.g., a porous glass, agarose, silica, cellulose or dextran gel), and compounds contained a mixture sample contact with the ligand. The preferred affinity chromatography is dye-ligand chromatography, which is exemplified as CM Affi-Gel Blue gel, DEAE Affi-Gel Blue gel (Bio-Rad Laboratories), or MIMETIC Red, Blue, Orange, Yellow or Green (Affinity Chromatography Ltd, Freeport, Great Britain). In particular, CM Affi-Gel Blue is preferable, which may contain Cibacron Blue F3GA dye covalently bound to a CM Bio-Gel A gel. The CM Bio-Gel A gel is a carboxy-terminal agarose gel, and this support is coupled with an amino-terminal ligand, protein or spacer arm. Conveniently, before loading of an eluate, the affinity chromatography column is equilibrated with an aqueous buffer solution of neutral pH, preferably, a phosphate buffer of about pH 7.2. The elution is carried out by a method known in the art using an aqueous buffer solution, preferably, a phosphate buffer of about pH 7.2. The phosphate buffer is preferably a 1 M sodium chloride-containing buffer. In case of carrying out elution with this buffer, an elution solution may be directly applied without an additional treatment to the second chromatography step, hydrophobic interaction chromatography column. The application of affinity chromatography as a first chromatography step allows for the effective removal of components of the culture medium (phenol red, etc.).

The hydrophobic interaction chromatography should be carried out on gels with hydrophobic, suitably aliphatic or aromatic, charge-free ligands attached to various commercially available matrices. The ligands can be coupled to the matrix by conventional coupling techniques giving charge-free ligands. The most common suitable example of such a technique is the glycidyl-ether coupling procedure. In another technique, an agarose matrix is first activated with glycidoxypropyltrimethoxy silane in water, and the ligands are then immobilized on the matrix in the alcohol. In yet another suitable technique, an agarose matrix is first activated with a bis-epoxide such as 1,4-butanediol diglycidyl ether. The obtained epoxy-activated gel can be coupled to a ligand such as aminoalkyl or alkyl mercaptan. Further available techniques are 1,1-carbonyldiimidazole activation and divinylsulfone activation. The gels obtained by the aforementioned techniques are charge-free within the entire pH range. The aliphatic ligand may be a straight alkyl such as propyl, butyl, pentyl, hexyl, heptyl or octyl, a branched alkyl such as iso- or neoalkyl, or oligoethylene glycol. The aromatic ligand is preferably a phenyl. The matrix can be selected from a group of strongly hydrophilic matrices, for example, an agarose matrix such as a Sepharose® matrix, an organic polymer matrix such as TSK-GEL, or a highly porous organic polymer matrix. The matrix is preferably an agarose matrix. Suitable agarose matrices in the present invention are Sepharose matrix sold by Amersham Biosciences (Uppsala, Sweden), Bio-Gel A sold by Bio-Rad Laboratories (Brussels, Belgium), and Minileak® sold by Kem-En-Tec A/S (Copenhagen, Denmark). Preferably, the matrix is cross-linked allowing for a fast flow (FF) and thereby high production capacity. More preferably, the hydrophobic interaction chromatography of the present invention is carried out on a Phenyl Sepharose 6 FF gel sold by Amersham Biosciences or a Butyl Sepharose 4 FF gel. If necessary, prior to the hydrophobic interaction chromatography step, a salt may be added to the eluted fractions to improve the conductivity of the fractions. Then, hTPO is eluted from the hydrophobic interaction chromatography column using a low ionic strength buffer. In an embodiment of the present invention, without such pretreatment, an eluate obtained in the affinity chromatography step was directly loaded onto the next hydrophobic interaction chromatography column. At the hydrophobic interaction chromatography step, hTPO is bound to the resin, and the impurities flow through the column or are removed by washing of the column, thereby allowing for the effective removal of most impurities.

The reverse phased chromatography is based on the separation of compounds according to their hydrophobic properties using a polar mobile phase and a nonpolar stationary phase (chemically bonded phase). The preferred reverse phase matrix includes C4 resins (Amersham Biosciences), and porous resins Oligo R2® and Oligo R3® (PerSeptive Biosystems, Inc., Framingham, Mass.). The typical solvent systems include water-ethanol, water-acetonitrile, water-tetrahydrofuran and hexylene glycol mixtures, and elution is carried out with a suitable concentration gradient of the solvent system by a conventionally known method. Preferably, the eluted fractions are immediately diluted with phosphate buffer to prevent the denaturation of proteins. Preferably, the reverse phased chromatography step in the purification process is carried out using a C4 reverse phase matrix. More preferably, the solvent system uses a gradient of the water-ethanol mixture. In an embodiment of the present invention, an eluant obtained by reverse phased chromatography using an ethanol concentration gradient was found to have a high purity of over 98%, resulting in almost a complete removal of the impurities contained in an elute obtained by the prior step hydrophobic interaction chromatography.

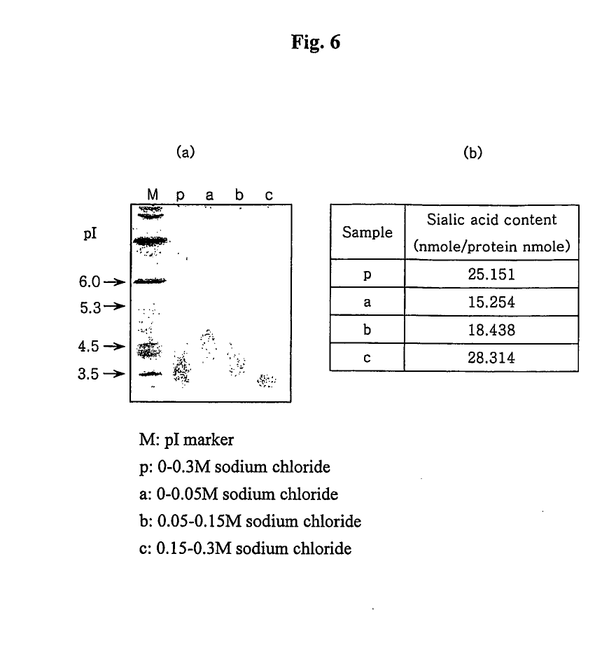

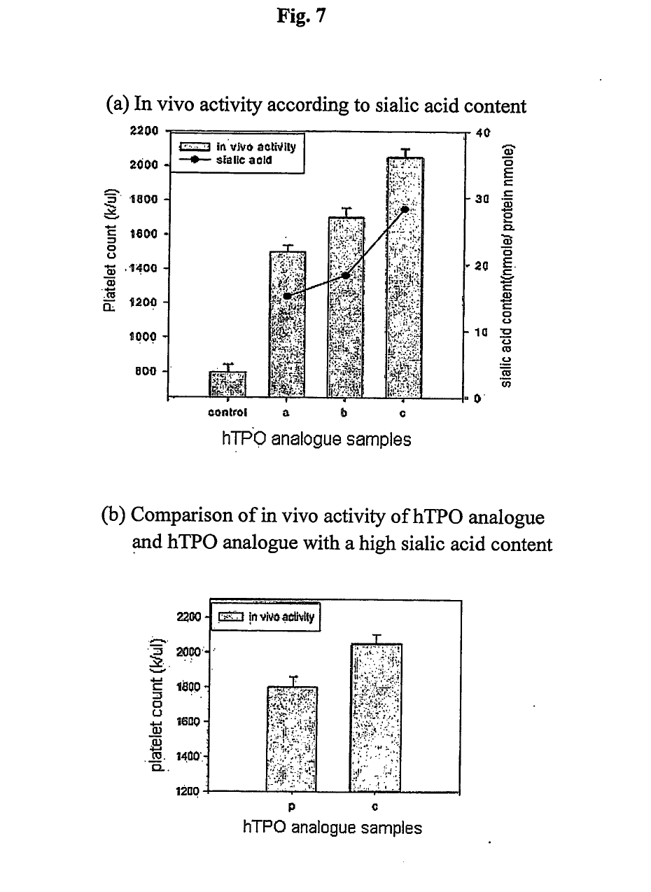

The anion exchange chromatography is typically carried out using a medium containing an insoluble particle support derivatized with a tertiary or quaternary amine group (e.g., diethylamnoethyl, triethylaminoethyl, benzyl-diethylaminoethyl). Suitable support includes cellulose, agarose, dextran and polystyrene beads. Preferably, the support is derivatized with the triethylaminoethyl group. Suitable anion exchange matrices include Q Sepharose® (Amersham Biosciences), Macro-Prep® Q (Bio-Rad Laboratories), Q-HyperD® (BioSepra, Inc., Marborough, Mass.), Fractogel EMD-TMAE 650 (Merck). Prior to the loading of an eluate onto an anion exchange column, the column may be conveniently equilibrated with an aqueous buffer solution of pH 6.0 to 8.0. Elution may be carried out using an aqueous buffer solution, and preferably, an acetate buffer having a pH ranging from about 4.5 to 6.5, by a conventionally known method. Alternatively, elution may be carried out by using a sodium phosphate buffer in a concentration gradient. Preferably, hTPO bound to an anion exchange chromatography column is eluted with a concentration gradient of sodium chloride, thereby allowing hTPO to be eluted according to its sialic acid contents. Sodium chloride may be used at a gradient of below 0.5 M, and preferably, below 0.3 M. When a higher gradient of sodium chloride was used, hTPO with a higher sialic acid content was eluted, and the results are given in FIGS. 6a and 6b, in which hTPO in the eluted fractions obtained by using the NaCl concentration gradient and its sialic acid contents are shown. As shown in FIGS. 6a and 6b, hTPO eluted with a gradient of 0.15 M to 0.3M NaCl was found to have the highest sialic acid content. The sialic acid content was determined by quantitative and qualitative analysis for N-acetylneuraminic acid and N-glyconeuraminic acid by isoelectric focusing. In addition, the purified hTPO was evaluated for the in vivo biological activity according to its sialic acid contents. As a result, when hTPO has an increased sialic acid content, platelet levels increased (Example 5 and FIG. 7).

Therefore, in a further aspect, the present invention provides a fraction containing hTPO with a high content of sialic acid by the process for purifying hTPO from an hTPO-containing biological fluid, comprising the steps of (a) subjecting the biological fluid to affinity chromatography; (b) subjecting the eluate obtained at step (a) to hydrophobic interaction chromatography; (c) subjecting the eluate obtained at step (b) to reverse phased chromatography; and (d) subjecting the eluate obtained at step (c) to anion exchange chromatography and collecting hTPO eluted selectively from the column by a 0.15-0.3M sodium chloride.

The term “hTPO with high sialic acid content”, as used herein, is intended to mean hTPO that is eluted from the aforementioned anion exchange chromatography column by a 0.15-0.3M sodium chloride gradient and has a pI of 4.0 and below.

hTPO purified by the chromatography steps may be further purified by gel filtration chromatography to remove aggregates in the eluate from the anion exchange chromatography column. The preferred matrix includes agarose, polyacrylamide or cross-linked beads of other polymers. More preferably, the matrix is Sephacryl (e.g., Sephacryl® S-200 HR or S-300 HR), Sephadex (e.g., Sephadex G50) or Superdex (e.g., Superdex® 200PG or Superdex 75), which are sold by Amersham Biosciences. Also, gel filtration matrices (e.g, TSK Toyopearl HW55) sold by TOSO Haas GmbH (Stuttgart, Germany) or similar gels sold by other manufacturers can be used. Elution may be carried out using an aqueous buffer by a conventionally known method. Also, other elution buffer solutions can be used, which are known to elute components negatively affecting TPO's properties. In a preferred aspect, the gel filtration chromatography step of the process for purifying hTPO is carried out using Superdex 200PG.

When being analyzed by reverse phased HPLC and gel filteration HPLC, hTPO purified by the chromatography steps as described above was found to have a high purity of 98% or more (Example 3).

The present invention will be explained in more detail with reference to the following example in conjunction with the accompanying drawings. However, it will be apparent to one skilled in the art that the following example is provided only to illustrate the present invention, and the present invention is not limited to the example.

### EXAMPLE 1

### Large-scale Serum-free Culture Using Cell Factory

For mass production of hTPO analogues with additional N-linked glycosylation by substitution of both amino acid residues at 157 and 164 positions with asparagines, a serum-free Cell Factory culture was carried out through both seed culture and large-scale culture steps. In the primary seed culture, five vials (1×10⁷ cell/ml) of hTPO analogue-producing cell line (CHO dhfr-/pD40458, KCTC 0632BP) were taken out from a working cell bank stored in liquid nitrogen, and washed once with a seed culture medium (5% serum-containing DMEM/F12, Gibco BRL Co.). Each 30 ml of a seed culture medium supplemented with methotrexate (Sigma) was added into five 175 cm² T-flasks (Nalge Nunc International Corp., Naperville, Ill.), and the washed cells were inoculated in the flasks, followed by incubation in a CO₂ incubator (37° C., 5% CO₂). When cell growth reached sub-confluency, the cells were treated with a 0.25% trypsin-EDTA solution. The cells recovered from one 175 cm² T-flask were inoculated in four new 175 cm² T-flasks, each of which contains 30 ml of a fresh seed culture medium supplemented with methotrexate. After the cells were cultured under the same conditions as described above until cell growth reached sub-confluency, the recovered cells were again inoculated in three 10-stack cell factories (Nunc Cell Factory of Nalge Nunc International Corp., Naperville, Ill.) containing 2 L of a fresh seed culture medium.

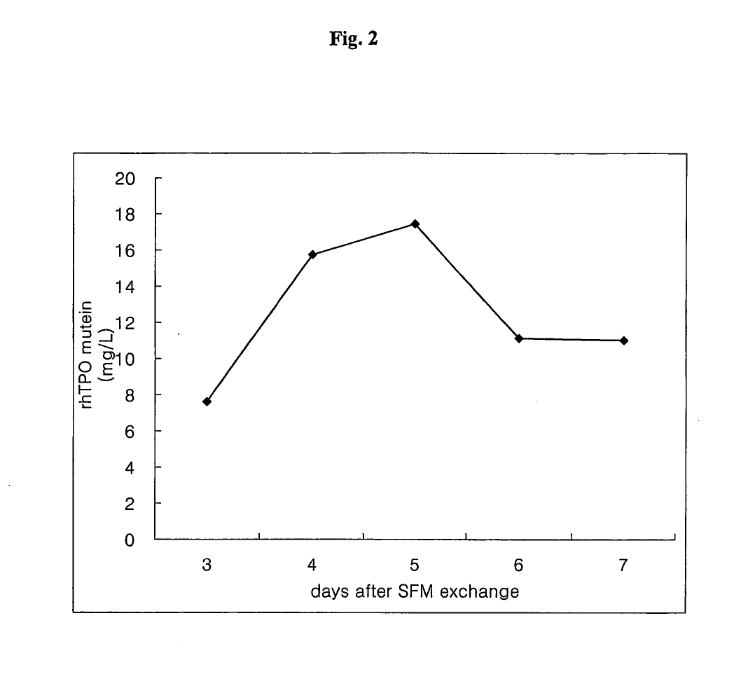

The cells recovered from three 10-stack cell factories were put into a Media bag (Stedim Inc., Concord, Calif.) containing 40 L of a fresh large-scale culture medium (1% serum-containing DMEM/F12, Gibco-BRL Co., Gaithersburg, Md.), and after mixing well, inoculated in five 40-stack cell factories at a density of 1.5×10⁵ cell/ml. 72 hrs after incubation, the cells were washed with PBS once, and the medium was exchanged to a serum-free DMEM/F12 supplemented with 0.5 mM butyric acid, yeastolate (Gibco-BRL Co.) and various amino acids, followed by incubation for 120 hrs in a CO₂ incubator (37° C., 5% CO₂). During the serum-free culture using the aforementioned serum-free medium, the cells were evaluated for expression levels of the hTPO analogue according to time. The results are given in FIG. 2. The highest expression level (20 mg/L) of the hTPO analogue was founded at 5 days after exchanging the serum-containing medium to the serum-free medium. Such an expression level was about 2-fold higher than an expression level (10 mg/L) in 10% serum-containing medium.

### EXAMPLE 2

### Purification of the hTPO Analogue

(a) Affinity Chromatography

A VS 150/500 column (Millipore, Bellerica, Mass.) was filled with 1 L of a CM Affi-Gel Blue resin (Bio-Rad Laboratories), and sufficiently washed with 10 L of buffer A (10 mM sodium phosphate, 150 mM sodium chloride, pH 7.2). 40 L of the culture supernatant prepared in Example 1 was passed through the column at a flow rate of 130 ml/min, and the flow through was monitored at 280 nm. After the cell supernatant completely passed through the column, the column was washed with buffer B (10 mM sodium phosphate, 2M urea, pH 7.2) until UV absorbance reached a basal level. Then, proteins including TPO, bound to the resin, were eluted with buffer C (10 mM sodium phosphate, 2M urea, 1M sodium chloride, pH 7.2).

(b) Hydrophobic Interaction Chromatography

A VS 90/500 column was filled with 800 ml of a Phenyl Sepharose FF resin (Amersham Biosciences), and sufficiently washed with 2 L of buffer C (10 mM sodium phosphate, 2M urea, 1M sodium chloride, pH 7.2). The eluted fractions obtained at the CM Affi-Gel Blue step were passed through the column at a flow rate of 43 ml/min, and the flow through was monitored at 280 nm. After the fractions were completely passed through the column, the column was washed with buffer C (10 mM sodium phosphate, 2M urea, 1M sodium chloride, pH 7.2) until UV absorbance reached a basal level. Then, proteins including TPO, bound to the resin, were eluted with buffer B (10 mM sodium phosphate, 2M urea, pH 7.2). After being supplemented with 20% ethanol, the resulting fractions were subjected to C4 reverse phased column chromatography.

(c) Reverse Phased Chromatography

A TR10/300 column (Amersham Biosciences) was filled with 23 ml of a C4 reverse phased resin (Amersham Pharmacia) of 15 μm in size, and equilibrated by washing with 50 mM sodium phosphate (pH 6.0), 20% ethanol. The eluted fractions (supplemented with 20% ethanol) obtained at the hydrophobic interaction chromatography step using the Phenyl Sepharose resin were loaded onto the column at a flow rate of 7 ml/min. The column was washed with 50 mM sodium phosphate (pH 6.0) and 40% ethanol. Then, the proteins bound to the resin were eluted with a 40%-80% ethanol gradient. Most of the expressed hTPO analogue was found to be eluted by the addition of about 70% ethanol. The fraction eluted from the column was diluted 10 times with 10 mM sodium phosphate buffer to prevent protein denaturation caused by organic solvents.

(d) Anion Exchange Chromatography

In order to separate the hTPO analogue according to its sialic acid content, the eluted fractions obtained at the reverse phased chromatography step were loaded onto an anion exchange chromatography Q column (Amersham Biosciences) at a flow rate of 10 ml/min. After being sufficiently washed with 10 mM sodium phosphate buffer, the column was eluted with 10 mM sodium phosphate buffer along with a 0-0.3 M sodium chloride gradient. The hTPO analogues with low sialic acid contents were found at the fractions eluted with below 0.15 M sodium chloride. In contrast, the hTPO analogue with high sialic acid contents was found at the fractions eluted with 0.15 M to 0.3 M sodium chloride.

(e) Gel Filtration Chromatography

hTPO with high sialic acid contents, obtained at the anion exchange chromatography step, was subjected to gel filtration chromatography to remove aggregates. In this step, TNT buffer (10 mM Tris, 150 mM sodium chloride, 0.01% Tween20), generally used as a buffer for final pharmaceutical formulations, was employed. XK50/100 (Amersharm Biosciences) was filled with 1.4 L of a gel filtration resin, Superdex 200PG (Amersharm Biosciences), and washed with 0.5 N NaOH and 0.5 N HCl. Then, 7 L of TNT buffer was passed through the column for one day to eliminate endotoxin from the resin. After loading an elution solution onto the column at a flow rate of 8 ml/min, and eluates were collected using a fraction collector (Amersham Biosciences). Fractions containing only hTPO being present in the monomer form were put together, filtered with a 0.22 μm membrane, aliquotted into vials for freeze-drying, and freeze-dried to allow for long-term storage.

### EXAMPLE 3

### Assays

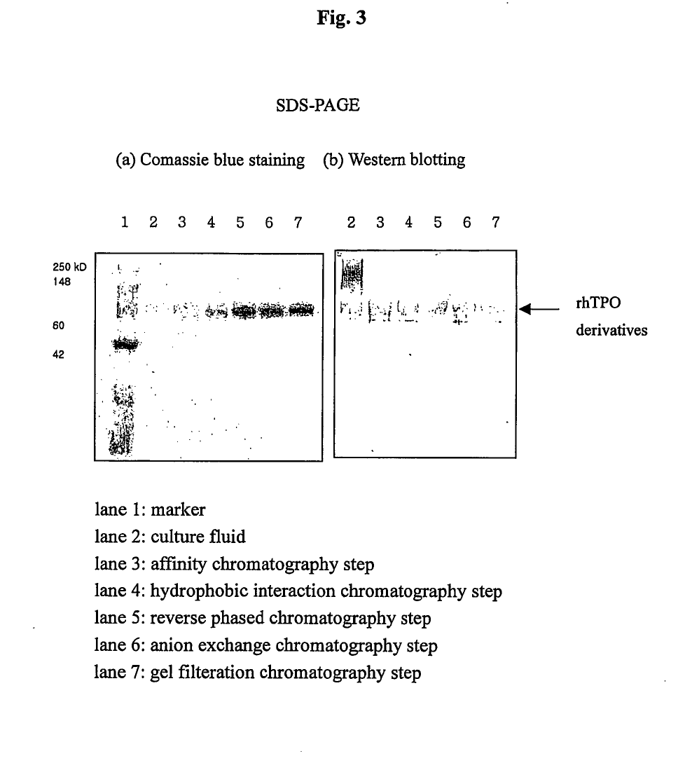

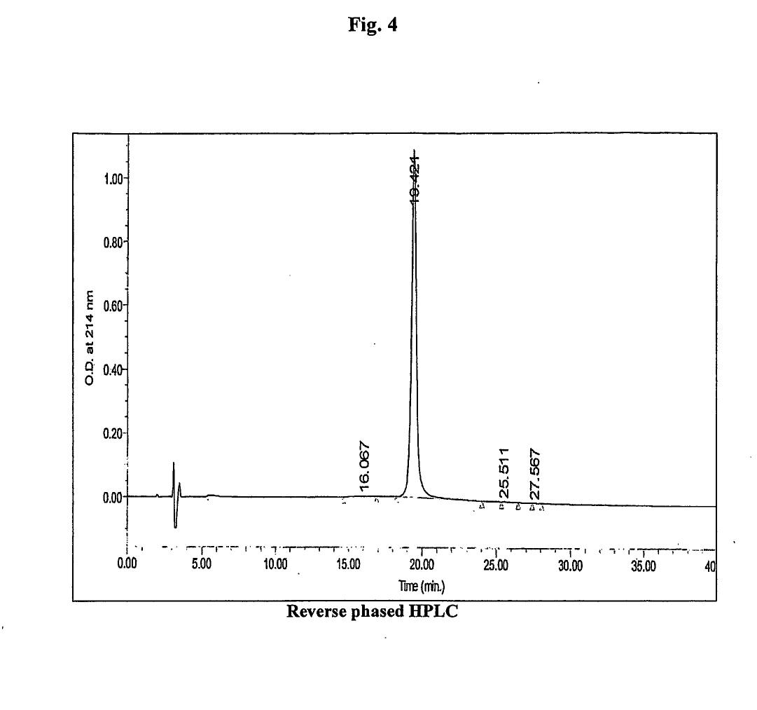

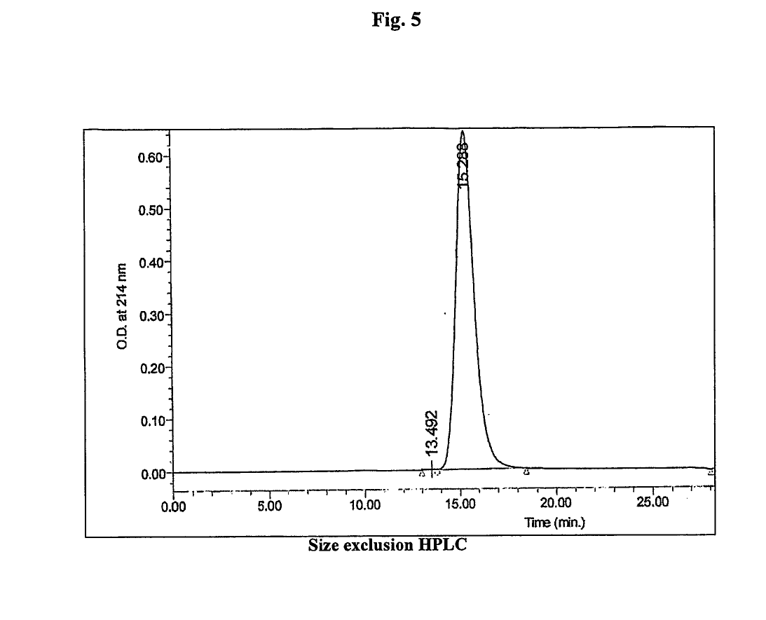

The eluates obtained at each chromatography step were electrophoresed on a 16% polyacrylamide gel (Invitorgen) under reducing conditions, and purified hTPO was identified by Coomassie blue staining and Western blotting. The results are given in FIGS. 3a and 3b. Also, the final purified product was analyzed by reverse phased HPLC (FIG. 4). As a result, the purified hTPO analogue was found to have a purity of over 99%. Further, an analysis by size exclusion HPLC (FIG. 5) demonstrated that over 98% of the purified hTPO analogue exists in a monomer form.

### EXAMPLE 4

### Isoelectrofocusing Analysis and Sialic Acid Analysis of the Purified hTPO Analogue

6 μg of each purified sample, prepared in Example 2, was loaded onto IEF (isoelectrofocusing) gel (pH 3-7, Invitrogen), and the gel electrophoresis was performed at 100 V for 1 hr, 200 V for 30 min, and then 500 V for 15 min. After electrophoresis, the gel was immersed in a fixing solution for 30 min, and stained with Coomassie blue staining. As a result, the fractions eluted with the sodium chloride concentration gradient from the Q column have reduced pI values when the salt concentration is increased (FIG. 6a). Separately, sialic acid contents of hTPO were determined as follows. 0.4 ml 0.1 N HCl was added to a dried sample of 0.4 to 0.6 nmol, and the sample was incubated for 1 hr at 80° C. to allow for the hydrolysis of sialic acid. The resulting solution was dried in a Speed Vac, dissolved again in distilled water and dried again. A portion of the dried was analyzed on a Bio-LC DX-300 system (Dionex Corporation, Sunnyvale, Calif.), using a CarboPac PA1 column (4 mm in diameter; and 250 mm in length) and 100 mM NaOH containing 150 mM sodium acetate at a flow rate of 1 ml/min. Herein, N-acetylneuraminic acid and N-glyconeuraminic acid, frequently found in glycoproteins, were used as standard materials, and quantitative and qualitative analysis for the standard materials were carried out. The results are given in FIG. 6b. The lower the pI vaue of a protein was, the higher its sialic acid content was. These results indicate that the sialic acid content of a glycoprotein largely affects its pI value.

### EXAMPLE 5

### Evaluation of In Vivo Biological Activity of the hTPO Analogue

The in vivo activity was analyzed by determining the platelet numbers in mice administered with the hTPO analogue expressed in the animal cells. 7 week-old BALB/c female mice (Charles River, Japan) were first adapted to a new environment for one week in an animal-breeding room at 24±1° C. under 55% humidity and 12 hr illumination (7 a.m. to 7 p.m.). The mice were also bred in the same room during the in vivo activity test. The mice were randomly divided into groups, each of which was composed of 5 mice. All groups except one was administered with the hTPO analogue, and the one group which was not administered with the hTPO analogue was used as a control.

hTPO with different sialic acid contents, eluted from the Q column with a different salt concentration in Example 2, was evaluated for in vivo activity. The purified hTPO was subcutaneously administered once to the mice at a concentration of 10 μg/kg body weight. After 5 days, blood samples were collected. After anesthetizing the mice, whole blood was collected from the abdominal inferior vena cava, and transferred to EDTA-treated tubes. Platelet numbers in peripheral blood were counted using an automatic blood cell counter (Cell dyne, Abbott). The results are designated as mean±SE. The platelet numbers were increased with high sialic acid contents (FIG. 7a). According to the same method as described above, the hTPO drivative eluted with a 0-0.3 M sodium chloride gradient was compared with the hTPO analogue with a high content of sialic acid eluted with a 0.15-0.3 M sodium chloride gradient (FIG. 7b). As a result, there was a significant difference between the two eluates in the in vivo activity.

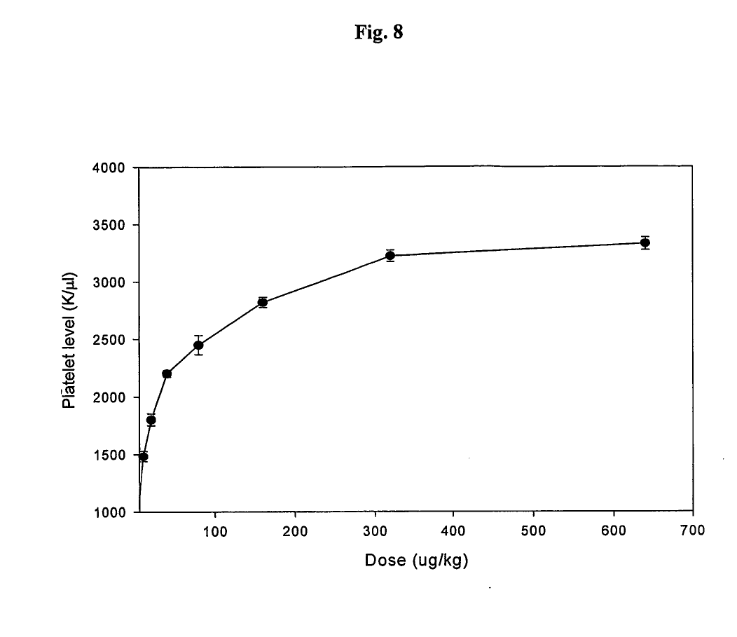

The fractions containing the hTPO analogue with a high content of sialic acid were subcutaneously administered once to the mice at various concentrations of 10, 20, 40, 80, 160, 320, 640 and 1280 μg/kg body weight. In vivo activity was analyzed according to the same method as described above. The results are given in FIG. 8, in which platelet numbers are plotted against the administration concentration of the hTPO analogue. The hTPO analogue promoted platelet production, and the highest platelet number was found on day 8. The hTPO analogue increased the platelet numbers in a dose-dependent manner.

### INDUSTRIAL APPLICABILITY

As described hereinbefore, hTPO can be produced by the culturing process of the present invention. In addition, hTPO with a high content of sialic acid can be obtained with a high purity by the purification process comprising various chromatography steps according to the present invention, while maintaining its in vivo biological activity. This highly pure hTPO with a high content of sialic acid is very useful in the medical field.

1. A process for producing a culture fluid containing human thrombopoietin (hTPO), comprising the steps of:
culturing a eukaryotic cell expressing hTPO in a 3-6.5% serum-containing medium;
subsequently culturing the cell in a 0.5-1.5% serum-containing medium; and
culturing the cell in a serum-free medium that is substantially free from serum.

2. The process as set forth in claim 1, wherein the eukaryotic cell is a Chinese hamster ovary (CHO) cell line.

3. The process as set forth in claim 2, wherein the CHO cell line is selected from the group consisting of CHO dhfr-/pD40434 (KCTC 0630BP), CHO dhfr-/pD40449 (KCTC 0631BP) and CHO dhfr-/pD40458 (KCTC 0632BP).

4. The process as set forth in claim 1, wherein the eukaryotic cell is inoculated in the 0.5-1.5% serum-containing medium at a density of 1.0×10⁴ to 1.0×10⁶ cells/ml.

5. The process as set forth in claim 4, wherein the eukaryotic cell is inoculated at a density of 1.5×10⁵ cells/ml.

6. The process as set forth in claim 1, wherein the serum-free medium is complemented with butyric acid and yeastolate.

7. A process for purifying human thrombopoietin (hTPO) from an hTPO-containing biological fluid, comprising the steps of:
(a) subjecting the biological fluid to affinity chromatography;
(b) subjecting the eluate obtained at step (a) to hydrophobic interaction chromatography;
(c) subjecting the eluate obtained at step (b) to reverse phased chromatography; and
(d) subjecting the eluate obtained at step (c) to anion exchange chromatography.

8. The process as set forth in claim 7, wherein the eluate obtained at step (c) is loaded onto an ionic exchange chromatography column, and hTPO eluted selectively from the column by a 0.15-0.3M sodium chloride gradient is collected.

9. The process as set forth in claim 7, further comprising a step of carrying out gel filtration chromatography after step (d).

10. The process as set forth in claim 7, wherein the hTPO-containing biological fluid is a culture supernatant from the culture fluid produced by the process of the claim 1.

11. The process as set forth in claim 7, wherein a column used in the affinity chromatography at step (a) is eluted with phosphate buffer containing 1 M sodium chloride.

12. The process as set forth in claim 7, wherein a column used in the reverse phased chromatography at step (c) is eluted with an ethanol gradient.

13. A fraction containing hTPO purified by the process of claim 8.
